(19) United States
(12) Patent Application Publication
(10) Pub. No.: US 2023/0129811 A1
(43) Pub. Date: Apr. 27, 2023

(54) REGENERATIVE T REGULATORY CELLS

(71) Applicant: CREATIVE MEDICAL TECHNOLOGIES INC., Phoenix, AZ (US)

(72) Inventors: Thomas ICHIM, Phoenix, AZ (US); Amit PATEL, Phoenix, AZ (US)

(73) Assignee: CREATIVE MEDICAL TECHNOLOGIES INC., Phoenix, AZ (US)

(21) Appl. No.: 18/045,433

(22) Filed: Oct. 10, 2022

Related U.S. Application Data
(60) Provisional application No. 63/270,678, filed on Oct. 22, 2021.

(51) Int. Cl. C12N 5/074

(52) U.S. Cl. CPC C12N 5/0696; C12N 2501/20; C12N 2501/40

(57) ABSTRACT

Disclosed are T regulatory cells endogenously occurring or generated in vitro which possess regenerative activity. In one embodiment said T regulatory cells are created by culture with derivatives of activated/enhanced mesenchymal stem cells. Said derivatives include apoptotic bodies, conditioned media, exosomes, microvesicles, proteins and various metabolites. In one embodiment said mesenchymal stem cells are activated with cytokines such as interferon gamma. In other embodiments are said mesenchymal stem cells are activated by ligation of toll like receptors. Cells of the invention are useful for treatment of autoimmune, degenerative, and combination of autoimmune and degenerative disease. Other uses of said regenerative T cells include treatment of heart failure, liver failure, stroke, and ischemic diseases.

### CROSS-REFERENCE TO RELATED APPLICATIONS

This application claims priority to U.S. Provisional Application Ser. No. 63/270,678, filed Oct. 22, 2021, which is hereby incorporated by reference in its entirety.

### FIELD OF THE INVENTION

The invention belongs to the area of immunotherapy. More specifically the invention pertains to the utilization of immunotherapy to induce regenerative processes in a mammal.

### BACKGROUND

The field of regenerative medicine has been characterized by numerous failures due to stem cell clinical trials missing endpoints. Primarily one of the disadvantages of utilizing mesenchymal stem cells is that due to their size and other properties, they poorly home to the organ of interest. In fact, a majority of mesenchymal stem cells actually become stuck in pulmonary capillaries.

New cell types capable of inducing regenerative activity are needed.

### SUMMARY

Preferred embodiments include methods of generating T cell possessing ability to suppress activation of another T cell while concurrently having ability to stimulate regenerative processes.

Preferred methods include embodiments wherein said T cell possesses the marker CD4.

Preferred methods include embodiments wherein said T cell is thymic derived.

Preferred methods include embodiments wherein said T cell is derived from a pluripotent stem cell.

Preferred methods include embodiments wherein said pluripotent stem cell is selected from a group of stem cells comprising of: a) inducible pluripotent stem cells; b) embryonic stem cells; c) parthenogenic derived stem cells; and d) somatic cell nuclear transfer derived stem cells.

Preferred methods include embodiments wherein said T cell expresses the transcription factor FoxP3.

Preferred methods include embodiments wherein said suppression of another T cell means inhibition of T cell proliferation.

Preferred methods include embodiments wherein said T cell proliferation is induced by an agent selected from one or more agents from a group comprising of: a) a mitogen; b) a cytokine; c) a molecule capable of activating T cell receptor in an antigen nonspecific manner; and d) a molecule capable of activating T cell receptor in an antigen specific manner.

Preferred methods include embodiments wherein said mitogen is conconavalin-A.

Preferred methods include embodiments wherein said mitogen is phytohemagluttinin.

Preferred methods include embodiments wherein said mitogen is pokeweed mitogen.

Preferred methods include embodiments wherein said mitogen is Galanthus nivalis Lectin.

Preferred methods include embodiments wherein said cytokine is interleukin-1 beta.

Preferred methods include embodiments wherein said cytokine is interleukin-2.

Preferred methods include embodiments wherein said cytokine is interleukin-3.

Preferred methods include embodiments wherein said cytokine is interleukin-4.

Preferred methods include embodiments wherein said cytokine is interleukin-7.

Preferred methods include embodiments wherein said cytokine is interleukin-10.

Preferred methods include embodiments wherein said cytokine is interleukin-12.

Preferred methods include embodiments wherein said cytokine is interleukin-18.

Preferred methods include embodiments wherein said cytokine is interleukin-17.

Preferred methods include embodiments wherein said cytokine is interleukin-20.

Preferred methods include embodiments wherein said cytokine is interleukin-35.

Preferred methods include embodiments wherein said cytokine is interferon alpha.

Preferred methods include embodiments wherein said cytokine is interferon beta.

Preferred methods include embodiments wherein said cytokine is interferon gamma.

Preferred methods include embodiments wherein said cytokine is interferon omega.

Preferred methods include embodiments wherein said molecule capable of activating said TCR in an antigen non-specific manner is an antibody.

Preferred methods include embodiments wherein said antibody is capable of ligating TCR.

Preferred methods include embodiments wherein said antibody is capable of ligating CD3.

Preferred methods include embodiments wherein said antibody is OKT3.

Preferred methods include embodiments wherein said molecule capable of activating said TCR in an antigen non-specific manner is a microbody.

Preferred methods include embodiments wherein said molecule capable of activating said TCR in an antigen non-specific manner is a bispecific antibody.

Preferred methods include embodiments wherein said molecule capable of activating said TCR in an antigen non-specific manner is a recombinant protein.

Preferred methods include embodiments wherein said molecule capable of activating said TCR in an antigen non-specific manner is an aptamer.

Preferred methods include embodiments wherein said molecule capable of activating said TCR in an antigen non-specific manner is a DNano particle.

Preferred methods include embodiments wherein said molecule capable of activating said TCR in an antigen specific manner is an autoantigen.

Preferred methods include embodiments wherein said suppression of another T cell means inhibition of T cell cytotoxic activity.

Preferred methods include embodiments wherein said T cell cytotoxic activity is mediated by Fas Ligand.

Preferred methods include embodiments wherein said T cell cytotoxic activity is mediated by TRAIL.

Preferred methods include embodiments wherein said T cell cytotoxic activity is mediated by perform.

Preferred methods include embodiments wherein said T cell cytotoxic activity is mediated by granzyme A.

Preferred methods include embodiments wherein said T cell cytotoxic activity is mediated by granzyme B.

Preferred methods include embodiments wherein said T cell cytotoxic activity is mediated by TNF-alpha.

Preferred methods include embodiments wherein said T cell cytotoxic activity is mediated by TNF-beta.

Preferred methods include embodiments wherein said T cell cytotoxic activity is mediated by induction of apoptosis.

Preferred methods include embodiments wherein said T cell cytotoxic activity is mediated by induction of necrosis.

Preferred methods include embodiments wherein said T cell cytotoxic activity is mediated by pyroptosis.

Preferred methods include embodiments wherein said T cell cytotoxic activity is mediated by necroptosis.

Preferred methods include embodiments wherein said T cell cytotoxic activity is mediated by alternation in mitochondrial membrane potential in the target cell.

Preferred methods include embodiments wherein said suppression of another T cell is downregulation of cytokine production of the target cell.

Preferred methods include embodiments wherein said cytokine production is induced by an agent selected from one or more agents from a group comprising of: a) a mitogen; b) a cytokine; c) a molecule capable of activating T cell receptor in an antigen nonspecific manner; and d) a molecule capable of activating T cell receptor in an antigen specific manner.

Preferred methods include embodiments wherein said mitogen is conconavalin-A.

Preferred methods include embodiments wherein said mitogen is phytohemagluttinin.

Preferred methods include embodiments wherein said mitogen is pokeweed mitogen.

Preferred methods include embodiments wherein said mitogen is Galanthus nivalis Lectin.

Preferred methods include embodiments wherein said cytokine is interleukin-1 beta.

Preferred methods include embodiments wherein said cytokine is interleukin-2.

Preferred methods include embodiments wherein said cytokine is interleukin-3.

Preferred methods include embodiments wherein said cytokine is interleukin-4.

Preferred methods include embodiments wherein said cytokine is interleukin-7.

Preferred methods include embodiments wherein said cytokine is interleukin-10.

Preferred methods include embodiments wherein said cytokine is interleukin-12.

Preferred methods include embodiments wherein said cytokine is interleukin-18.

Preferred methods include embodiments wherein said cytokine is interleukin-17.

Preferred methods include embodiments wherein said cytokine is interleukin-20.

Preferred methods include embodiments wherein said cytokine is interleukin-35.

Preferred methods include embodiments wherein said cytokine is interferon alpha.

Preferred methods include embodiments wherein said cytokine is interferon beta.

Preferred methods include embodiments wherein said cytokine is interferon gamma.

Preferred methods include embodiments wherein said cytokine is interferon omega.

Preferred methods include embodiments wherein said molecule capable of activating said TCR in an antigen non-specific manner is an antibody.

Preferred methods include embodiments wherein said antibody is capable of ligating TCR.

Preferred methods include embodiments wherein said antibody is capable of ligating CD3.

Preferred methods include embodiments wherein said antibody is OKT3.

Preferred methods include embodiments wherein said molecule capable of activating said TCR in an antigen non-specific manner is a microbody.

Preferred methods include embodiments wherein said molecule capable of activating said TCR in an antigen non-specific manner is a bispecific antibody.

Preferred methods include embodiments wherein said molecule capable of activating said TCR in an antigen non-specific manner is a recombinant protein.

Preferred methods include embodiments wherein said molecule capable of activating said TCR in an antigen non-specific manner is an aptamer.

Preferred methods include embodiments wherein said molecule capable of activating said TCR in an antigen non-specific manner is a DNano particle.

Preferred methods include embodiments wherein said molecule capable of activating said TCR in an antigen specific manner is an autoantigen.

Preferred methods include embodiments wherein said regenerative properties are ability to stimulate angiogenesis.

Preferred methods include embodiments wherein said angiogenesis is creation of collateral circulation.

Preferred methods include embodiments wherein said collateral circulation is used to treat ischemic conditions.

Preferred methods include embodiments wherein said ischemic conditions are associated with activation of hypoxia inducible factor-1.

Preferred methods include embodiments wherein said angiogenesis is associated with production of VEGF.

Preferred methods include embodiments wherein said angiogenesis is associated with mobilization of endothelial progenitor cells.

Preferred methods include embodiments wherein said angiogenesis is associated with mobilization of mesenchymal stem cells.

Preferred methods include embodiments wherein said angiogenesis is associated with mobilization of hematopoietic stem cells.

Preferred methods include embodiments wherein said regeneration involves activation of endogenous stem cells or progenitor cells.

Preferred methods include embodiments wherein said endogenous stem cells or progenitor cells are capable of differentiating into tissue that is injured or hypofunctional.

Preferred methods include embodiments wherein said endogenous stem cells or progenitor cells are mitotically quiescent in their basal state.

Preferred methods include embodiments wherein said endogenous stem cells or progenitor cells express aldehyde dehydrogenase.

Preferred methods include embodiments wherein said endogenous stem cells or progenitor cells express stem cell antigen-1.

Preferred methods include embodiments wherein said endogenous stem cells or progenitor cells express interleukin-1 receptor.

Preferred methods include embodiments wherein said endogenous stem cells or progenitor cells express interleukin-3 receptor.

Preferred methods include embodiments wherein said endogenous stem cells or progenitor cells express c-maf.

Preferred methods include embodiments wherein said endogenous stem cells or progenitor cells express interleukin-10 receptor.

Preferred methods include embodiments wherein said endogenous stem cells or progenitor cells express interleukin-11 receptor.

Preferred methods include embodiments wherein said endogenous stem cells or progenitor cells express wnt.

Preferred methods include embodiments wherein said T cells are extracted from a source selected from a group of sources comprising of: a) peripheral blood; b) mobilized peripheral blood; c) umbilical cord blood; d) menstrual blood; e) bone marrow; f) cerebral spinal fluid; and g) adipose tissue.

Preferred methods include embodiments wherein said T cell is a CD4 T cell.

Preferred methods include embodiments wherein said T cell obtained by culture of peripheral blood mononuclear cells in the presence of conditioned/enhanced media from an activated mesenchymal stem cell.

Preferred methods include embodiments wherein said activated mesenchymal stem cells is stimulated by exposure to a cytokine.

Preferred methods include embodiments wherein said cytokine activates the JAK/STAT pathway.

Preferred methods include embodiments wherein said cytokine is interferon gamma.

Preferred methods include embodiments wherein said cytokine is interferon alpha.

Preferred methods include embodiments wherein said cytokine is interferon beta.

Preferred methods include embodiments wherein said cytokine is interferon tau.

Preferred methods include embodiments wherein said cytokine is interleukin-1 beta.

Preferred methods include embodiments wherein said cytokine is tumor necrosis factor alpha.

Preferred methods include embodiments wherein said cytokine is tumor necrosis factor beta.

Preferred methods include embodiments wherein said cytokine is interleukin-6.

Preferred methods include embodiments wherein said cytokine is8 interleukin-8.

Preferred methods include embodiments wherein said cytokine is interleukin-9.

Preferred methods include embodiments wherein said cytokine is interleukin-11.

Preferred methods include embodiments wherein said cytokine is interleukin-17.

Preferred methods include embodiments wherein said cytokine is interleukin-18.

Preferred methods include embodiments wherein said cytokine is interleukin-21.

Preferred methods include embodiments wherein said cytokine is interleukin-23.

Preferred methods include embodiments wherein said cytokine is interleukin-27.

Preferred methods include embodiments wherein said cytokine is interleukin-33.

Preferred methods include embodiments wherein said mesenchymal stem cells are activated by treatment with an activator of an immune receptor.

Preferred methods include embodiments wherein said immune receptor activates immunotyrosine activation motifs.

Preferred methods include embodiments wherein said immune receptor activates NF-AT.

Preferred methods include embodiments wherein said immune receptor activates NF-kappa B.

Preferred methods include embodiments wherein said immune receptor activates STAT-3.

Preferred methods include embodiments wherein said immune receptor activates STAT-4.

Preferred methods include embodiments wherein said immune receptor activates janus activated kinase.

Preferred methods include embodiments wherein said immune receptor activates MAP-kinase.

Preferred methods include embodiments wherein said immune receptor is TLR. 1

Preferred methods include embodiments wherein said TLR-1 is activated by Pam3CSK4.

Preferred methods include embodiments wherein said immune receptor is TLR-2

Preferred methods include embodiments wherein said TLR-2 is activated by HKLM.

Preferred methods include embodiments wherein said immune receptor is TLR-3.

Preferred methods include embodiments wherein said TLR-3 is activated by Poly:IC.

Preferred methods include embodiments wherein said immune receptor is TLR-4.

Preferred methods include embodiments wherein said TLR-4 is activated by LPS.

Preferred methods include embodiments wherein said TLR-4 is activated by Buprenorphine.

Preferred methods include embodiments wherein said TLR-4 is activated by Carbamazepine.

Preferred methods include embodiments wherein said TLR-4 is activated by Fentanyl.

Preferred methods include embodiments wherein said TLR-4 is activated by Levorphanol.

Preferred methods include embodiments wherein said TLR-4 is activated by Methadone.

Preferred methods include embodiments wherein said TLR-4 is activated by Cocaine.

Preferred methods include embodiments wherein said TLR-4 is activated by Morphine.

Preferred methods include embodiments wherein said TLR-4 is activated by Oxcarbazepine.

Preferred methods include embodiments wherein said TLR-4 is activated by Oxycodone.

Preferred methods include embodiments wherein said TLR-4 is activated by Pethidine.

Preferred methods include embodiments wherein said TLR-4 is activated by Glucuronoxylomannan from Cryptococcus.

Preferred methods include embodiments wherein said TLR-4 is activated by Morphine-3-glucuronide.

Preferred methods include embodiments wherein said TLR-4 is activated by lipoteichoic acid.

Preferred methods include embodiments wherein said TLR-4 is activated by beta.-defensin 2.

Preferred methods include embodiments wherein said TLR-4 is activated by low molecular weight hyaluronic acid.

Preferred methods include embodiments wherein said low molecular weight hyaluronic acid has a molecular weight of <1000 kDa.

Preferred methods include embodiments wherein said low molecular weight hyaluronic acid has a molecular weight of <500 kDa.

Preferred methods include embodiments wherein said low molecular weight hyaluronic acid has a molecular weight of <250 kDa.

Preferred methods include embodiments wherein said low molecular weight hyaluronic acid has a molecular weight of <100 kDa.

Preferred methods include embodiments wherein said TLR-4 is activated by fibronectin EDA.

Preferred methods include embodiments wherein said TLR-4 is activated by snapin.

Preferred methods include embodiments wherein said TLR-4 is activated by tenascin C.

Preferred methods include embodiments wherein said immune receptor is TLR-5.

Preferred methods include embodiments wherein said TLR-5 is activated by flaggelin.

Preferred methods include embodiments wherein said immune receptor is TLR-6.

Preferred methods include embodiments wherein said TLR-6 is activated by FSL-1.

Preferred methods include embodiments wherein said immune receptor is TLR-7.

Preferred methods include embodiments wherein said TLR-7 is activated by imiquimod.

Preferred methods include embodiments wherein said immune receptor is TLR-8.

Preferred methods include embodiments wherein said TLR-8 is activated by ssRNA40/LyoVec.

Preferred methods include embodiments wherein said immune receptor is TLR-9.

Preferred methods include embodiments wherein said TLR-9 is activated by a CpG oligonucleotide.

Preferred methods include embodiments wherein said TLR-9 is activated by ODN2006.

Preferred methods include embodiments wherein said TLR-9 is activated by Agatolimod.

Preferred methods include embodiments wherein said TLR-9 is activated by ODN2007.

Preferred methods include embodiments wherein said TLR-9 is activated by ODN1668.

Preferred methods include embodiments wherein said TLR-9 is activated by ODN1826.

Preferred methods include embodiments wherein said TLR-9 is activated by ODN BW006.

Preferred methods include embodiments wherein said TLR-9 is activated by ODN D SL01.

Preferred methods include embodiments wherein said TLR-9 is activated by ODN 2395.

Preferred methods include embodiments wherein said TLR-9 is activated by ODN M362.

Preferred methods include embodiments wherein said TLR-9 is activated by ODN SL03.

Preferred methods include embodiments wherein said T cells express CD123.

Preferred methods include embodiments wherein said T cells express IL-10.

Preferred methods include embodiments wherein said T cells express leukemia inhibitory factor.

Preferred methods include embodiments wherein said T cells are capable of treating an ischemic disease.

Preferred methods include embodiments wherein said ischemic disease is congestive heart failure.

Preferred methods include embodiments wherein said ischemic disease is stroke.

Preferred methods include embodiments wherein said ischemic disease is liver failure.

Preferred methods include embodiments wherein said ischemic disease is peripheral artery disease.

Preferred methods include embodiments wherein said ischemic disease is critical limb ischemia.

Preferred methods include embodiments wherein said ischemic disease is intestinal ischemia.

Preferred methods include embodiments wherein said T cells are capable of treating a degenerative disease.

Preferred methods include embodiments wherein said T cells express OCT-4.

Preferred methods include embodiments wherein said T cells express NANOG.

Preferred methods include embodiments wherein said T cells express SOX-2.

Preferred methods include embodiments where in the potency of the conditioned/enhanced media is measured by HGF levels.

### DETAILED DESCRIPTION OF THE INVENTION

The invention, in some embodiments, teaches the utilization of T cells are regenerative cells whereby said cells possess immune regulatory and repair properties. It is known that a cardinal feature of the immune system, is allowing for recognition and elimination of pathological threats, while selectively ignoring antigens that belong to the body. Traditionally, autoimmune conditions or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia.

The current approach described teaches generation of T cells and T regulatory cells which possess immunological and regenerative properties.

The invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as Wharton's Jelly. In some embodiments manipulation of stem cell “potency” is disclosed through hypoxic manipulation, growth on non-xenogeneic conditions, as well as addition of epigenetic modulators.

The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, a population of stem cells are utilized to induce reprogramming of T cells to endow a phenotype associated with regeneration. These “Regenerative T cells” possess expression of the initerleukin-3 receptor, which is one of several distinguishing features. For the practice of the invention, MSC are used to reprogramme immune cells in order to endow cardiac regenerative activity can be utilized as was previously performed in clinical trials with non-selected MSC. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

“Administering” as used herein, refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab).sub.2, as well as single chain antibodies and humanized antibodies. The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. .kappa. and .lamda. light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means, in accordance with the present invention, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

“Allogeneic” refers to a graft derived from a different animal of the same species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Other specific types of cancer include cinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

“Costimulatory ligand” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, CD7, B7-1 (CD80), B7-2 (CD86), programmed death (PD) L1, PD-L2, 4-1BB ligand, OX40 ligand, inducible costimulatory ligand (ICOS-L), intercellular adhesion molecule (ICAM), CD30 ligand, CD40, CD70, CD83, human leukocyte antigen G (HLA-G), MHC class I chain-related protein A (MICA), MHC class I chain-related protein B (MICB), herpes virus entry mediator (HVEM), lymphotoxin beta receptor, 3/TR6, immunoglobulin-like transcript (ILT) 3, ILT4, an agonist or antibody that binds Toll ligand receptor and a ligand that specifically binds with B7-H3. A co-stimulatory ligand includes, without limitation, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, tumor necrosis factor superfamily member 14 (TNFSF14 or LIGHT), natural killer cell receptor C (NKG2C), B7-H3, and a ligand that specifically binds with CD83.

The term “CD137” refers to a TNFR-family member with costimulatory function. CD137 is also called 4-1BB or TNFSFR9. It was originally identified as an inducible molecule expressed on activated mouse and human CD8+ and CD4+ T-cells. CD137 signaling regulates T-cell proliferation and survival, particularly within the T-cell memory pool and can upregulate Bcl-X.sub.L anti-apoptotic protein expression and supports CD8+ T-cell expansion.

“Cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used here a ligand is a molecule such as an antigen that forms a complex with its appropriate or complimentary biomolecule, an anti-ligand such as an antibody. An anti-ligand is a molecule having a high binding specificity for its ligand such as a protein binding to its appropriate hormone or a vitamin , an antibody binding to its appropriate antigenic determinant, oligonucleotide sequence binding to its complementary fragment of RNA or DNA The anti-ligand may be monovalent or polyvalent. Anti-ligand-apheresis is defined here as the specific removal of blood plasma and/or its components using an apparatus followed by returning the remainder blood back into the circulation of the patient. Some of the examples include but are not limited to immunopheresis, plasmapheresis, leukapheresis, pateletpheresis and renal dialysis machines. Apheresis, anti-ligand-apheresis and immunopheresis may be used interchangeably. Synbodies are defined as synthetic antibodies. Procedures for generation of synbodies are described in the following and incorporated by reference [1-6]. These terms have the same meaning herein as conventionally used in the art. Housing as used here is defined as a casing or an enclosure.

The term “lymphocyte” means T cells, B cells, NK cells, and Lymphokine Activated Killer (LAK) cells. T-lymphocytes possess T-cell receptors, B-lymphocytes, possess B cell receptors and produce antibodies, Tumor Infiltrating Lymphocytes (TIL) are isolated from tumors and possess some degree of reactivity towards the tumor, cytotoxic T lymphocytes (CTL) are lymphocytes of the CD8 lineage usually and possess ability to kill cells through perforin and/or granzymes. CTL isolation means are described in numerous references including U.S. Pat. No. 6,805,861 and U.S. Pat. No. 6,531,451. Any one lymphocyte produces one type of TCR or antibody. Each TCR or antibody has specificity for one particular epitope, or antigen binding site, on its cognate antigen. Specific TCRs or antibodies are encoded by genes that are formed from the rearrangement of DNA in a lymphocyte stem cell that encodes the constant (“C”), joining (“J”), variable (“V”) regions, and possibly diversity (“D”) regions of the TCR or antibody. Mammals typically possess one-hundred thousand to one-hundred million lymphocytes of different specificities. Upon stimulation of lymphocytes by an antigen, those lymphocytes specific for the antigen undergo clonal amplification. T lymphocytes are formed in the bone marrow, migrate to and mature in the thymus and then enter the peripheral blood and lymphatic circulation. T lymphocytes are subdivided into three distinct types of cells: helper T cells, suppressor T cells, and cytotoxic T cells. T lymphocytes, unlike B lymphocytes, do not produce antibody molecules, but express a heterodimeric cell surface receptor that recognizes peptide fragments of antigenic proteins that are attached to proteins of the major histocompatibility complex (MHC) and expressed on the surfaces of target cells. T lymphocytes include tumor-infiltrating lymphocytes. Cytotoxic T lymphocytes (CTL) are well known in the art and are typically of the CD3+, CD8+, CD4− phenotype. They typically lyse cells that display fragments of foreign antigens associated with class I MHC molecules on their cell surfaces. CTL typically recognize normal cells expressing antigens after infection by viruses or other pathogens; and tumor cells that have undergone transformation and are expressing mutated proteins or are over-expressing normal proteins. Natural Killer (NK) cells are well known in the art. NK cells are a subset of lymphocytes active in the immune system and representing an average 15% of mononuclear cells in human peripheral blood. Among the surface markers used to identify human NK cells is a receptor binding with low affinity to the Fc fragment of IgG antibodies, such as Fc-.gamma. receptor III or CD16 antigen. NK cells have been demonstrated to play an important role in vivo in the defense against tumors, tumor metastases, virus infection, and to regulate normal and malignant hematopoiesis. Lymphokine-activated killer (LAK) cells are well known in the art and are a cytotoxic population of cells which are capable of lysing autologous tumor cells and NK-cell resistant tumor cell lines. Precursors of LAK cells belong to the subpopulation of “null” lymphocytes that bear neither T nor B cell surface markers. In the human these precursor cells are widely found in peripheral blood, lymph nodes, bone marrow and the thoracic duct. Purification of LAK cells, and their generation are described in U.S. Pat. Nos. 5,002,879, 4,849,329 and 4,690,915.

The term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, such as a mammal, or cells thereof whether in vitro or in situ, amenable to the methods described herein. In certain non-limiting embodiments, the patient, subject or individual is a human.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-.beta., and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an artificial APC [7], a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). Examples of subjects include humans, dogs, cats, mice, rats, and transgenic species thereof.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

For the practice of the invention, MSC are used to reprogramme immune cells in order to endow cardiac regenerative activity can be utilized as was previously performed in clinical trials with non-selected MSC. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In some embodiments, dendritic cells are generated which possess tolerogenic activity and said dendritic cells are pulsed with cardiac antigens. Said cardiac antigens include the myosin heavy chain [8]. Said dendritic cells can be made tolerogenic by culture in cytokines such as IL-10 or they can be further made tolerogenic by culture with regenerative cells. In one embodiment said regenerative cells are mesenchymal stem cells. Generation of clinical grade dendritic cells is described in the following papers which are incorporated by reference [9-133].

Mesenchymal stem cells (MSCs) are adult stem cells with self-renewing abilities[134] and have been shown to differentiate into a wide range of tissues including mesoderm- and nonmesoderm-derived[134, 135], such as hepatocytes[136-141]. MSCs are capable of entering and maintaining satellite cell niches, particularly in hematopoiesis[142, 143], and are key in tissue repair and regeneration, aging, and regulating homeostasis[144-147]. In the case of heart failure, MSCs can aid in regeneration of cardiac tissue[148-154], and their interactions with the immune system[155-161] have potential as adjuvants during organ transplants[162], including cardiac transplantation[163].

MSCs were discovered in 1970 by Friedenstein et al[164] who demonstrated that bone marrow (BM) contained both hematopoietic stem cells (HSCs), which are non-plastic adherent, and a population of a more rare adherent cell. The adherent cells were able to form single cell colonies and were referred to as stromal cells. Those stromal cells, which are capable of self-renewal and expansion in culture are now referred to as mesenchymal stem cells (MSCs). Friedenstein was the first to show that MSCs could differentiate into mesoderm and to demonstrate their importance in controlling the hematopoietic niche [165].

In the 1980s, more research on MSCs found that they could differentiate into muscle, cartilage, bone and adipose-derived cells [166]. Caplan et al showed that MSCs are responsible for bone and cartilage regeneration induced by local cuing and genetic potential[167].

In the 1990s, Pittenger et al isolated MSCs from bone marrow and found that they retained their multilineage potential after expanding into selectively differentiated adipocytic, chondrocytic, or osteocytic lineages[135]. Likewise, Kopen et al showed that bone marrow MSCs differentiated into neural cells when exposed to the brain microenvironment[168]. In 1999, Petersen et al found that bone marrow-derived stem cells could be a source of hepatic oval cells in a rat model[169]. Specifically, they used male to female bone marrow transplant and subsequently induced blockade of hepatocyte proliferation by administration of a hepatotoxin followed by partial hepatectomy. As previously described, this procedure stimulates proliferation of LPC or “reserve cells” which generate new hepatocytes, such cells having previously identified as oval cells. Subsequent to the hepatectomy, Y chromosome, dipeptidyl peptidase IV enzyme, and L21-6 antigen were used to identify the newly generated oval cells, and their hepatocytic progeny to be of bone marrow origin.

The first decade of the 21st century saw a surge of research on MSCs, leading to a greater understanding of their nature and of the cellular process behind regeneration [134, 146, 147]. In 2005, Teratani et al identified growth factors allowing hepatic fate-specification in mice and showed that embryonic stem cells could differentiate into functional hepatocytes [170]. A unique property of MSC is their apparent hypoimmunogenicity and immune modulatory activity

, which is present in MSC derived from various sources [172]. This is believed to account for the ability to achieve therapeutic effects in an allogeneic manner. Allogeneic bone marrow derived MSC have been used by academic investigators with clinical benefit treatment of diseases such as graft versus host (GVHD) [173-178], osteogenesis imperfecta [179], Hurler syndrome, metachromatic leukodystrophy [180], and acceleration of hematopoietic stem cell engraftment [181-183]. The company Athersys has successfully completed Phase I safety studies using allogeneic bone marrow MSCs is now in efficacy finding clinical trials (Phase II and Phase III) for Multiple Sclerosis, Crohn's Disease, and Graft Versus Host Disease using allogeneic bone marrow derived MSC. Intravenous administration of allogeneic MSCs by Osiris was also reported to induce a statistically significant improvement in cardiac function in a double-blind study [184].

Currently there several MSC-based therapies that have received governmental approvals including Prochymal™ which was registered in Canada and New Zealand for treatment of graft versus host disease [185, 186]. Although in terms of clinical translation bone marrow MSC are the most advanced, several other sources of MSC are known which possess various properties that may be useful for specific conditions. Bone marrow is also a source for hematopoietic stem cells (HSCs), which have also been used for cardiac regeneration. Likewise, human placenta is an easily accessible source of abundant MSCs, which can be differentiated in vitro. Finally, MSCs with tissue regenerative abilities can also be isolated from adipose tissue and induced to hepatocytes in large numbers.

In some embodiments of the invention reprogrammed immune cells are administered with MSC for treatment of heart failure. The discussion below provides examples of the use of MSC in heart failure, which may be useful for one of skill in the art to combine MSC with reprogramemd immune cells

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In one embodiment, bone marrow MSC lots are generated, means of generating BM MSC are known in the literature and examples are incorporated by reference.

In one embodiment BM-MSC are generated as follows
1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10.mu.L BM and diluting it 1/50- 1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 mL conical tubes (BD Catalog #352070), 17 mL Ficoll-Paque.™. PLUS (Catalog #07907/07957) is pipetted into each tube. About 23 mL of the diluted BM from step 3 was carefully layered on top of the Ficoll-Paque.™. PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma:Ficoll-Paque.™. PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult.RTM.-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′107 cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′106 cells per ml in 175 cm2 tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′106 per 175 cm2. Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA , washed with DPBS +2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody . Confluent MSC in 175 cm2 flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199 +1% human serum albumin (HSA; American Red Cross, Washington D.C., USA). MSCs harvested from each 10-flask set were stored for up to 4 h at 4° C. and combined at the end of the harvest. A total of 2-10′ 106 MSC/kg were resuspended in M199 +1% HSA and centrifuged at 460 g for 10 min at 20° C. Cell pellets were resuspended in fresh M199 +1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

Within the context of the invention, exosomes and microparticles may be used interchangeably. Exosomes from MSC may be generated from a mesenchymal stem cell conditioned medium (MSC-CM). Said exosomes are used in the context of the invention to reprogram immunocytes ex vivo or in vivo. Said particle may be isolated for example by being separated from non-associated components based on any property of the particle. For example, the particle may be isolated based on molecular weight, size, shape, composition or biological activity. The conditioned medium may be filtered or concentrated or both during, prior to or subsequent to separation. For example, it may be filtered through a membrane, for example one with a size or molecular weight cut-off. It may be subject to tangential force filtration or ultrafiltration. Filtration of conditioned media is described in the following and incorporated by reference [187]. For example, filtration with a membrane of a suitable molecular weight or size cutoff. The conditioned medium, optionally filtered or concentrated or both, may be subject to further separation means, such as column chromatography. For example, high performance liquid chromatography (HPLC) with various columns may be used. The columns may be size exclusion columns or binding columns. One or more properties or biological activities of the particle may be used to track its activity during fractionation of the mesenchymal stem cell conditioned medium (MSC-CM). As an example, light scattering, refractive index, dynamic light scattering or UV-visible detectors may be used to follow the particles. For example, a therapeutic activity such as antirheumatic activity may be used to track the activity during fractionation. In one embodiment antirheumatic activity is assessed by ability to inhibit TNF-alpha production from stimulated monocytes or monocytic lineage cell such as macrophages or dendritic cells.

In one aspect of the invention MSC are cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes.

MSC can be prepared from a variety of tissues, such as bone marrow cells [188-194], umbilical cord tissue [195-197], peripheral blood [198-200], amniotic membrane [201], amniotic fluid, mobilized peripheral blood [202], adipose tissue [203, 204], endometrium and other tissues. When tissue sources of MSC are used said tissue isolates from which the Reprogrammed immune cells are isolated comprise a mixed populations of cells. Reprogrammed immune cells constitute a very small percentage in these initial populations. They must be purified away from the other cells before they can be expanded in culture sufficiently to obtain enough cells for therapeutic applications.

The choice of formulation for administering reprogrammed immune cells for a given application will depend on a variety of factors. Prominent among these will be the species of subject, the nature of the disorder, dysfunction, or disease being treated and its state and distribution in the subject, the nature of other therapies and agents that are being administered, the optimum route for administration of the reprogrammed immune cells, survivability of reprogrammed immune cells via the route, the dosing regimen, and other factors that will be apparent to those skilled in the art. In particular, for instance, the choice of suitable carriers and other additives will depend on the exact route of administration and the nature of the particular dosage form, for example, liquid dosage form (e.g., whether the composition is to be formulated into a solution, a suspension, gel or another liquid form, such as a time release form or liquid-filled form).

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic Reprogrammed immune cells. Various embodiments of the invention comprise measures to increase cell survival and/or to overcome problems posed by barriers to seeding and/or growth.

Examples of compositions comprising reprogrammed immune cells include liquid preparations, including suspensions and preparations for intramuscular or intravenous administration (e.g., injectable administration), such as sterile suspensions or emulsions. Such compositions may comprise an admixture of Reprogrammed immune cells with a suitable carrier, diluent, or excipient such as sterile water, physiological saline, glucose, dextrose, or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Compositions of the invention often are conveniently provided as liquid preparations, e.g., isotonic aqueous solutions, suspensions, emulsions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues.

Various additives often will be included to enhance the stability, sterility, and isotonicity of the compositions, such as antimicrobial preservatives, antioxidants, chelating agents, and buffers, among others. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents that delay absorption, for example, aluminum monostearate, and gelatin. According to the present invention, however, any vehicle, diluent, or additive used would have to be compatible with the cells.

Reprogrammed immune cells solutions, suspensions, and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose) may also be present. In some embodiments of the invention, treatment of viral associated cardiac damage is disclosed using immune cells that have been reprogrammed with regenerative cells. Some patients hospitalized for COVID-19 have had increased levels of cardiac enzyme or markers that indicate their hearts are at least temporarily damaged. Also, cardiac damage is more common in patients who have severe COVID-19 disease.

Typically, the compositions will be isotonic, i.e., they will have the same osmotic pressure as blood and lacrimal fluid when properly prepared for administration.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or cell stabilizer can be employed to increase the life of reprogrammed immune cells compositions. If such preservatives are included, it is well within the purview of the skilled artisan to select compositions that will not affect the viability or efficacy of the reprogrammed immune cells.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells utilized in practicing the present invention in the required amount of the appropriate solvent with various amounts of the other ingredients, as desired.

In some embodiments, reprogrammed immune cells are formulated in a unit dosage injectable form, such as a solution, suspension, or emulsion. Pharmaceutical formulations suitable for injection of Reprogrammed immune cells typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

For any composition to be administered to an animal or human, and for any particular method of administration, it is preferred to determine therefore: toxicity, such as by determining the lethal dose (LD) and LD50 in a suitable animal model, e.g., rodent such as mouse or rat; and, the dosage of the composition(s), concentration of components therein, and timing of administering the composition(s), which elicit a suitable response. Such determinations do not require undue experimentation from the knowledge of the skilled artisan, this disclosure, and the documents cited herein. And, the time for sequential administrations can be ascertained without undue experimentation.

In some embodiments Reprogrammed immune cells are encapsulated for administration, particularly where encapsulation enhances the effectiveness of the therapy, or provides advantages in handling and/or shelf life. Encapsulation in some embodiments where it increases the efficacy of the cell mediated immunosuppression may, as a result, also reduce the need for immunosuppressive drug therapy.

Also, encapsulation in some embodiments provides a barrier to a subject's immune system that may further reduce a subject's immune response to the Reprogrammed immune cells (which generally are not immunogenic or are only weakly immunogenic in allogeneic transplants), thereby reducing any graft rejection or inflammation that might occur upon administration of the cells.

In a variety of embodiments where reprogrammed immune cells are administered in admixture with cells of another type, which are more typically immunogenic in an allogeneic or xenogeneic setting, encapsulation may reduce or eliminate adverse host immune responses to the non-reprogrammed immune cells cells and/or GVHD that might occur in an immunocompromised host if the admixed cells are immunocompetent and recognize the host as non-self.

Reprogrammed immune cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval.

A wide variety of materials may be used in various embodiments for microencapsulation of Reprogrammed immune cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of Reprogrammed immune cells are known to those of skill in the art and are described, for example, in Chang, P., et al., 1999; Matthew, H. W., et al., 1991; Yanagi, K., et al., 1989; Cal Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of Reprogrammed immune cells.

Certain embodiments incorporate Reprogrammed immune cells into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, Reprogrammed immune cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

Pharmaceutical compositions of the invention may be prepared in many forms that include tablets, hard or soft gelatin capsules, aqueous solutions, suspensions, and liposomes and other slow-release formulations, such as shaped polymeric gels. Oral liquid pharmaceutical compositions may be in the form of, for example, aqueous or oily suspensions, solutions, emulsions, syrups, or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives. An oral dosage form may be formulated such that cells are released into the intestine after passing through the stomach. Such formulations are described in U.S. Pat. No. 6,306,434 and in the references contained therein.

Pharmaceutical compositions suitable for rectal administration can be prepared as unit dose suppositories. Suitable carriers include saline solution and other materials commonly used in the art.

For administration by inhalation, cells can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, a means may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, cells may be administered via a liquid spray, such as via a plastic bottle atomizer.

### REFERENCES

ADDIN EN.REFLIST 1. Diehnelt, C. W., et al., Discovery of high-affinity protein binding ligands—backwards. PLoS One, 2010. 5(5): p. e10728.
2. Liu, R., et al., Generating DNA synbodies from previously discovered peptides. Chembiochem, 2011. 12(12): p. 1813-7.
3. Gupta, N., et al., Engineering a synthetic ligand for tumor necrosis factor-alpha. Bioconjug Chem, 2011. 22(8): p. 1473-8.
4. Domenyuk, V., et al., A technology for developing synbodies with antibacterial activity. PLoS One, 2013. 8(1): p. e54162.
5. Lainson, J. C., et al., Conjugation Approach To Produce a Staphylococcus aureus Synbody with Activity in Serum. Bioconjug Chem, 2015. 26(10): p. 2125-32.
6. Gupta, N., et al., Whole-Virus Screening to Develop Synbodies for the Influenza Virus. Bioconjug Chem, 2016. 27(10): p. 2505-2512.
7. Maus, M. V., et al., Ex vivo expansion of polyclonal and antigen-specific cytotoxic T lymphocytes by artificial APCs expressing ligands for the T-cell receptor, CD28 and 4-IBB. Nat Biotechnol, 2002. 20(2): p. 143-8.
8. DeBerge, M., et al., Monocytes prime autoreactive T cells after myocardial infarction. Am J Physiol Heart Circ Physiol, 2020. 318(1): p. H116-H123.
9. Nestle, F. O., et al., Vaccination of melanoma patients with peptide- or tumor lysate-pulsed dendritic cells. Nat Med, 1998. 4(3): p. 328-32.
10. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.
11. Wang, F., et al., Phase I trial of a MART-1 peptide vaccine with incomplete Freund's adjuvant for resected high-risk melanoma. Clin Cancer Res, 1999. 5(10): p. 2756-65.
12. Thurner, B., et al., Vaccination with mage-3A1 peptide-pulsed mature, monocyte-derived dendritic cells expands specific cytotoxic T cells and induces regression of some metastases in advanced stage IV melanoma. J Exp Med, 1999. 190(11): p. 1669-78.
13. Thomas, R., et al., Immature human monocyte-derived dendritic cells migrate rapidly to draining lymph nodes after intradermal injection for melanoma immunotherapy. Melanoma Res, 1999. 9(5): p. 474-81.
14. Mackensen, A., et al., Phase I study in melanoma patients of a vaccine with peptide-pulsed dendritic cells generated in vitro from CD34(+) hematopoietic progenitor cells. Int J Cancer, 2000. 86(3): p. 385-92.
15. Panelli, M. C., et al., Phase 1 study in patients with metastatic melanoma of immunization with dendritic cells presenting epitopes derived from the melanoma-associated antigens MART-1 and gp100. J Immunother, 2000. 23(4): p. 487-98.
16. Schuler-Thurner, B., et al., Mage-3 and influenza-matrix peptide-specific cytotoxic T cells are inducible in terminal stage HLA-A2.1+melanoma patients by mature monocyte-derived dendritic cells. J Immunol, 2000. 165(6): p. 3492-6.
17. Lau, R., et al., Phase I trial of intravenous peptide-pulsed dendritic cells in patients with metastatic melanoma. J Immunother, 2001. 24(1): p. 66-78.
18. Banchereau, J., et al., Immune and clinical responses in patients with metastatic melanoma to CD34(+) progenitor-derived dendritic cell vaccine. Cancer Res, 2001. 61(17): p. 6451-8.
19. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.
20. Palucka, A. K., et al., Single injection of CD34+ progenitor-derived dendritic cell vaccine can lead to induction of T-cell immunity in patients with stage IV melanoma. J Immunother, 2003. 26(5): p. 432-9.
21. Bedrosian, I., et al., Intranodal administration of peptide pulsed mature dendritic cell vaccines results in superior CD8+ T-cell function in melanoma patients. J Clin Oncol, 2003. 21(20): p. 3826-35.
22. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
23. Hersey, P., et al., Phase I/II study of treatment with dendritic cell vaccines in patients with disseminated melanoma. Cancer Immunol Immunother, 2004. 53(2): p. 125-34.
24. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.
25. Palucka, A. K., et al., Spontaneous proliferation and type 2 cytokine secretion by CD4+T cells in patients with metastatic melanoma vaccinated with antigen pulsed dendritic cells. J Clin Immunol, 2005. 25(3): p. 288-95.
26. Banchereau, J., et al., Immune and clinical outcomes in patients with stage IV melanoma vaccinated with peptide pulsed dendritic cells derived from CD34+ progenitors and activated with type I interferon. J Immunother, 2005. 28(5): p. 505-16.
27. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.
28. Salcedo, M., et al., Vaccination of melanoma patients using dendritic cells loaded with an allogeneic tumor cell lysate. Cancer Immunol Immunother, 2006. 55(7): p. 819-29.
29. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.
30. Escobar, A., et al., Dendritic cell immunizations alone or combined with low doses of interleukin-2 induce specific immune responses in melanoma patients. Clin Exp Immunol, 2005. 142(3): p. 555-68.
31. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.
32. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17(4): p. 563-70.
33. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.
34. Nakai, N., et al., Vaccination of Japanese patients with advanced melanoma with peptide, tumor lysate or both peptide and tumor lysate-pulsed mature, monocyte-derived dendritic cells. J Dermatol, 2006. 33(7): p. 462-72.
35. Palucka, A. K., et al., Dendritic cells loaded with killed allogeneic melanoma cells can induce objective clinical responses and MART-1 specific CD8+ T-cell immunity. J Immunother, 2006. 29(5): p. 545-57.
36. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.
37. Guo, J., et al., Intratumoral injection of dendritic cells in combination with local hyperthermia induces systemic antitumor effect in patients with advanced melanoma. Int J Cancer, 2007. 120(11): p. 2418-25.
38. O'Rourke, M. G., et al., Dendritic cell immunotherapy for stage IV melanoma. Melanoma Res, 2007. 17(5): p. 316-22.
39. Bercovici, N., et al., Analysis and characterization of antitumor T-cell response after administration of dendritic cells loaded with allogeneic tumor lysate to metastatic melanoma patients. J Immunother, 2008. 31(1): p. 101-12.
40. Hersey, P., et al., Phase I/II study of treatment with matured dendritic cells with or without low dose IL-2 in patients with disseminated melanoma. Cancer Immunol Immunother, 2008. 57(7): p. 1039-51.
41. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10 -1082 promoter genotype as predictor of disease progression. J Transl Med, 2008. 6: p. 6.
42. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.
43. Redman, B. G., et al., Phase Ib trial assessing autologous, tumor-pulsed dendritic cells as a vaccine administered with or without IL-2 in patients with metastatic melanoma. J Immunother, 2008. 31(6): p. 591-8.
44. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.
45. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.
46. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53(1): p. 40-7.
47. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.
48. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.
49. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.
50. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.
51. Ribas, A., et al., Multicenter phase II study of matured dendritic cells pulsed with melanoma cell line lysates in patients with advanced melanoma. J Transl Med, 2010. 8: p. 89.
52. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
53. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.
54. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60(2): p. 249-60.
55. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.
56. Steele, J. C., et al., Phase I/II trial of a dendritic cell vaccine transfected with DNA encoding melan A and gp100 for patients with metastatic melanoma. Gene Ther, 2011. 18(6): p. 584-93.
57. Kim, D. S., et al., Immunotherapy of malignant melanoma with tumor lysate pulsed autologous monocyte-derived dendritic cells. Yonsei Med J, 2011. 52(6): p. 990-8.
58. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.
59. Dillman, R. O., et al., Tumor stem cell antigens as consolidative active specific immunotherapy: a randomized phase II trial of dendritic cells versus tumor cells in patients with metastatic melanoma. J Immunother, 2012. 35(8): p. 641-9.
60. Dannull, J., et al., Melanoma immunotherapy using mature DCs expressing the constitutive proteasome. J Clin Invest, 2013. 123(7): p. 3135-45.
61. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.
62. Stift, A., et al., Dendritic cell vaccination in medullary thyroid carcinoma. Clin Cancer Res, 2004. 10(9): p. 2944-53.
63. Kuwabara, K., et al., Results of a phase I clinical study using dendritic cell vaccinations for thyroid cancer. Thyroid, 2007. 17(1): p. 53-8.
64. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.
65. Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.
66. Yamanaka, R., et al., Vaccination of recurrent glioma patients with tumour lysate pulsed dendritic cells elicits immune responses: results of a clinical phase I/II trial. Br J Cancer, 2003. 89(7): p. 1172-9.
67. Yu, J. S., et al., Vaccination with tumor lysate-pulsed dendritic cells elicits antigen-specific, cytotoxic T-cells in patients with malignant glioma. Cancer Res, 2004. 64(14): p. 4973-9.
68. Yamanaka, R., et al., Tumor lysate and IL-18 loaded dendritic cells elicits Thi response, tumor-specific CD8+ cytotoxic T cells in patients with malignant glioma. J Neurooncol, 2005. 72(2): p. 107-13.
69. Yamanaka, R., et al., Clinical evaluation of dendritic cell vaccination for patients with recurrent glioma: results of a clinical phase I/II trial. Clin Cancer Res, 2005. 11(11): p. 4160-7.
70. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.
71. Walker, D. G., et al., Results of a phase I dendritic cell vaccine trial for malignant astrocytoma: potential interaction with adjuvant chemotherapy. J Clin Neurosci, 2008. 15(2): p. 114-21.
72. Leplina, O. Y., et al., Use of interferon-alpha-induced dendritic cells in the therapy of patients with malignant brain gliomas. Bull Exp Biol Med, 2007. 143(4): p. 528-34.
73. De Vleeschouwer, S., et al., Postoperative adjuvant dendritic cell-based immunotherapy in patients with relapsed glioblastoma multiforme. Clin Cancer Res, 2008. 14(10): p. 3098-104.
74. Ardon, H., et al., Adjuvant dendritic cell-based tumour vaccination for children with malignant brain tumours. Pediatr Blood Cancer, 2010. 54(4): p. 519-25.
75. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.
76. Okada, H., et al., Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29(3): p. 330-6.
77. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.
78. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.
79. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
80. Iwami, K., et al., Peptide pulsed dendritic cell vaccination targeting interleukin-13 receptor alpha2 chain in recurrent malignant glioma patients with HLA-A*24/A*02 allele. Cytotherapy, 2012. 14(6): p. 733-42.
81. Fong, B., et al., Monitoring of regulatory T cell frequencies and expression of CTLA-4 on T cells, before and after DC vaccination, can predict survival in GBM patients. PLoS One, 2012. 7(4): p. e32614.
82. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.
83. Phuphanich, S., et al., Phase I trial of a multi-epitope-pulsed dendritic cell vaccine for patients with newly diagnosed glioblastoma. Cancer Immunol Immunother, 2013. 62(1): p. 125-35.
84. Akiyama, Y., et al., alpha-type-1 polarized dendritic cell-based vaccination in recurrent high-grade glioma: a phase I clinical trial. BMC Cancer, 2012. 12: p. 623.
85. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.
86. Shah, A. H., et al., Dendritic cell vaccine for recurrent high-grade gliomas in pediatric and adult subjects: clinical trial protocol. Neurosurgery, 2013. 73(5): p. 863-7.
87. Reichardt, V. L., et al., Idiotype vaccination using dendritic cells after autologous peripheral blood stem cell transplantation for multiple myeloma—a feasibility study. Blood, 1999. 93(7): p. 2411-9.
88. Lim, S. H. and R. Bailey-Wood, Idiotypic protein pulsed dendritic cell vaccination in multiple myeloma. Int J Cancer, 1999. 83(2): p. 215-22.
89. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.
90. Reichardt, V. L., et al., Idiotype vaccination of multiple myeloma patients using monocyte-derived dendritic cells. Haematologica, 2003. 88(10): p. 1139-49.
91. Guardino, A. E., et al., Production of myeloid dendritic cells (DC) pulsed with tumor-specific idiotype protein for vaccination of patients with multiple myeloma. Cytotherapy, 2006. 8(3): p. 277-89.
92. Lacy, M. Q., et al., Idiotype-pulsed antigen-presenting cells following autologous transplantation for multiple myeloma may be associated with prolonged survival. Am J Hematol, 2009. 84(12): p. 799-802.
93. Yi, Q., et al., Optimizing dendritic cell-based immunotherapy in multiple myeloma: intranodal injections of idiotype-pulsed CD40 ligand-matured vaccines led to induction of type-1 and cytotoxic T-cell immune responses in patients. Br J Haematol, 2010. 150(5): p. 554-64.
94. Rollig, C., et al., Induction of cellular immune responses in patients with stage-I multiple myeloma after vaccination with autologous idiotype-pulsed dendritic cells. J Immunother, 2011. 34(1): p. 100-6.
95. Zahradova, L., et al., Efficacy and safety of Id-protein-loaded dendritic cell vaccine in patients with multiple myeloma—phase II study results. Neoplasma, 2012. 59(4): p. 440-9.
96. Timmerman, J. M., et al., Idiotype-pulsed dendritic cell vaccination for B-cell lymphoma: clinical and immune responses in 35 patients. Blood, 2002. 99(5): p. 1517-26.
97. Maier, T., et al., Vaccination of patients with cutaneous T-cell lymphoma using intranodal injection of autologous tumor-lysate-pulsed dendritic cells. Blood, 2003. 102(7): p. 2338-44.
98. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.
99. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.
100. Li, L., et al., Immunotherapy for patients with acute myeloid leukemia using autologous dendritic cells generated from leukemic blasts. Int J Oncol, 2006. 28(4): p. 855-61.
101. Roddie, H., et al., Phase I/II study of vaccination with dendritic-like leukaemia cells for the immunotherapy of acute myeloid leukaemia. Br J Haematol, 2006. 133(2): p. 152-7.
102. Litzow, M. R., et al., Testing the safety of clinical-grade mature autologous myeloid DC in a phase I clinical immunotherapy trial of CML. Cytotherapy, 2006. 8(3): p. 290-8.
103. Westermann, J., et al., Vaccination with autologous non-irradiated dendritic cells in patients with bcr/abl+chronic myeloid leukaemia. Br J Haematol, 2007. 137(4): p. 297-306.
104. Hus, I., et al., Vaccination of B-CLL patients with autologous dendritic cells can change the frequency of leukemia antigen-specific CD8+ T cells as well as CD4+ CD25+ FoxP3+ regulatory T cells toward an antileukemia response. Leukemia, 2008. 22(5): p. 1007-17.
105. Palma, M., et al., Development of a dendritic cell-based vaccine for chronic lymphocytic leukemia. Cancer Immunol Immunother, 2008. 57(11): p. 1705-10.
106. Van Tendeloo, V. F., et al., Induction of complete and molecular remissions in acute myeloid leukemia by Wilms' tumor 1 antigen-targeted dendritic cell vaccination. Proc Natl Acad Sci U S A, 2010. 107(31): p. 13824-9.
107. Iwashita, Y., et al., A phase I study of autologous dendritic cell-based immunotherapy for patients with unresectable primary liver cancer. Cancer Immunol Immunother, 2003. 52(3): p. 155-61.
108. Lee, W. C., et al., Vaccination of advanced hepatocellular carcinoma patients with tumor lysate-pulsed dendritic cells: a clinical trial. J Immunother, 2005. 28(5): p. 496-504.
109. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.
110. Palmer, D. H., et al., A phase II study of adoptive immunotherapy using dendritic cells pulsed with tumor lysate in patients with hepatocellular carcinoma. Hepatology, 2009. 49(1): p. 124-32.
111. El Ansary, M., et al., Immunotherapy by autologous dendritic cell vaccine in patients with advanced HCC. J Cancer Res Clin Oncol, 2013. 139(1): p. 39-48.
112. Tada, F., et al., Phase I/II study of immunotherapy using tumor antigen-pulsed dendritic cells in patients with hepatocellular carcinoma. Int J Oncol, 2012. 41(5): p. 1601-9.
113. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.
114. Hirschowitz, E. A., et al., Autologous dendritic cell vaccines for non-small-cell lung cancer. J Clin Oncol, 2004. 22(14): p. 2808-15.
115. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.
116. Yannelli, J. R., et al., The large scale generation of dendritic cells for the immunization of patients with non-small cell lung cancer (NSCLC). Lung Cancer, 2005. 47(3): p. 337-50.
117. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.
118. Antonia, S. J., et al., Combination of p53 cancer vaccine with chemotherapy in patients with extensive stage small cell lung cancer. Clin Cancer Res, 2006. 12(3 Pt 1): p. 878-87.
119. Perrot, I., et al., Dendritic cells infiltrating human non-small cell lung cancer are blocked at immature stage. J Immunol, 2007. 178(5): p. 2763-9.
120. Hirschowitz, E. A., et al., Immunization of NSCLC patients with antigen pulsed immature autologous dendritic cells. Lung Cancer, 2007. 57(3): p. 365-72.
121. Baratelli, F., et al., Pre-clinical characterization of GMP grade CCL21-gene modified dendritic cells for application in a phase I trial in non-small cell lung cancer. J Transl Med, 2008. 6: p. 38.
122. Hegmans, J. P., et al., Consolidative dendritic cell-based immunotherapy elicits cytotoxicity against malignant mesothelioma. Am J Respir Crit Care Med, 2010. 181(12): p. 1383-90.
123. Um, S. J., et al., Phase I study of autologous dendritic cell tumor vaccine in patients with non-small cell lung cancer. Lung Cancer, 2010. 70(2): p. 188-94.
124. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.
125. Perroud, M. W., Jr., et al., Mature autologous dendritic cell vaccines in advanced non-small cell lung cancer: a phase I pilot study. J Exp Clin Cancer Res, 2011. 30: p. 65.
126. Skachkova, O. V., et al., Immunological markers of anti-tumor dendritic cells vaccine efficiency in patients with non-small cell lung cancer. Exp Oncol, 2013. 35(2): p. 109-13.
127. Hernando, J. J., et al., Vaccination with autologous tumour antigen-pulsed dendritic cells in advanced gynaecological malignancies: clinical and immunological evaluation of a phase I trial. Cancer Immunol Immunother, 2002. 51(1): p. 45-52.
128. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.
129. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.
130. Kandalaft, L. E., et al., A Phase I vaccine trial using dendritic cells pulsed with autologous oxidized lysate for recurrent ovarian cancer. J Transl Med, 2013. 11: p. 149.
131. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6(B): p. 955-964.
132. Rong, Y., et al., A phase I pilot trial of MUC1 -peptide-pulsed dendritic cells in the treatment of advanced pancreatic cancer. Clin Exp Med, 2012. 12(3): p. 173-80.
133. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.
134. Jackson, L., et al., Adult mesenchymal stem cells: differentiation potential and therapeutic applications. J Postgrad Med, 2007. 53(2): p. 121-7.
135. Pittenger, M. F., et al., Multilineage potential of adult human mesenchymal stem cells. Science, 1999. 284(5411): p. 143-7.
136. Banas, A., et al., Rapid hepatic fate specification of adipose-derived stem cells and their therapeutic potential for liver failure. J Gastroenterol Hepatol, 2009. 24(1): p. 70-7.
137. Lee, K. D., et al., In vitro hepatic differentiation of human mesenchymal stem cells. Hepatology, 2004. 40(6): p. 1275-84.
138. Cho, K. A., et al., Mesenchymal stem cells showed the highest potential for the regeneration of injured liver tissue compared with other subpopulations of the bone marrow. Cell Biol Int, 2009. 33(7): p. 772-7.
139. Hong, S. H., et al., In vitro differentiation of human umbilical cord blood-derived mesenchymal stem cells into hepatocyte-like cells. Biochem Biophys Res Commun, 2005. 330(4): p. 1153-61.
140. Ishikawa, T., et al., Stem cells for hepatic regeneration: the role of adipose tissue derived mesenchymal stem cells. Curr Stem Cell Res Ther, 2010. 5(2): p. 182-9.
141. Seo, M. J., et al., Differentiation of human adipose stromal cells into hepatic lineage in vitro and in vivo. Biochem Biophys Res Commun, 2005. 328(1): p. 258-64.
142. Crisan, M., et al., A perivascular origin for mesenchymal stem cells in multiple human organs. Cell Stem Cell, 2008. 3(3): p. 301-13.
143. Tavian, M. and B. Peault, Embryonic development of the human hematopoietic system. Int J Dev Biol, 2005. 49(2-3): p. 243-50.
144. Peault, B., et al., Stem and progenitor cells in skeletal muscle development, maintenance, and therapy. Mol Ther, 2007. 15(5): p. 867-77.
145. Aggarwal, S. and M. F. Pittenger, Human mesenchymal stem cells modulate allogeneic immune cell responses. Blood, 2005. 105(4): p. 1815-22.
146. Caplan, A. I., Adult mesenchymal stem cells for tissue engineering versus regenerative medicine. J Cell Physiol, 2007. 213(2): p. 341-7.
147. Chamberlain, G., et al., Concise review: mesenchymal stem cells: their phenotype, differentiation capacity, immunological features, and potential for homing. Stem Cells, 2007. 25(11): p. 2739-49.
148. Banas, A., et al., IFATS collection: in vivo therapeutic potential of human adipose tissue mesenchymal stem cells after transplantation into mice with liver injury. Stem Cells, 2008. 26(10): p. 2705-12.
149. Kharaziha, P., et al., Improvement of liver function in liver cirrhosis patients after autologous mesenchymal stem cell injection: a phase I-II clinical trial. Eur J Gastroenterol Hepatol, 2009. 21(10): p. 1199-205.
150. Kuo, T. K., et al., Stem cell therapy for liver disease: parameters governing the success of using bone marrow mesenchymal stem cells. Gastroenterology, 2008. 134(7): p. 2111-21, 2121 e1-3.
151. Chang, Y. J., et al., Mesenchymal stem cells facilitate recovery from chemically induced liver damage and decrease liver fibrosis. Life Sci, 2009. 85(13-14): p. 517-25.
152. Lu, L. L., et al., Isolation and characterization of human umbilical cord mesenchymal stem cells with hematopoiesis-supportive function and other potentials. Haematologica, 2006. 91(8): p. 1017-26.
153. Mohamadnejad, M., et al., Phase 1 trial of autologous bone marrow mesenchymal stem cell transplantation in patients with decompensated liver cirrhosis. Arch Iran Med, 2007. 10(4): p. 459-66.
154. Terai, S., et al., Improved liver function in patients with liver cirrhosis after autologous bone marrow cell infusion therapy. Stem Cells, 2006. 24(10): p. 2292-8.
155. Chang, C. J., et al., Placenta-derived multipotent cells exhibit immunosuppressive properties that are enhanced in the presence of interferon-gamma. Stem Cells, 2006. 24(11): p. 2466-77.
156. Iyer, S. S. and M. Rojas, Anti-inflammatory effects of mesenchymal stem cells: novel concept for future therapies. Expert Opin Biol Ther, 2008. 8(5): p. 569-81.
157. Nauta, A. J. and W. E. Fibbe, Immunomodulatory properties of mesenchymal stromal cells. Blood, 2007. 110(10): p. 3499-506.
158. Uccelli, A., V. Pistoia, and L. Moretta, Mesenchymal stem cells: a new strategy for immunosuppression? Trends Immunol, 2007. 28(5): p. 219-26.
159. Wolbank, S., et al., Dose-dependent immunomodulatory effect of human stem cells from amniotic membrane: a comparison with human mesenchymal stem cells from adipose tissue. Tissue Eng, 2007. 13(6): p. 1173-83.
160. Wolf, D. and A. M. Wolf, Mesenchymal stem cells as cellular immunosuppressants.

Lancet, 2008. 371(9624): p. 1553-4.
161. Shi, M., Z. W. Liu, and F. S. Wang, Immunomodulatory properties and therapeutic application of mesenchymal stem cells. Clin Exp Immunol, 2011. 164(1): p. 1-8.
162. Sordi, V. and L. Piemonti, Therapeutic plasticity of stem cells and allograft tolerance. Cytotherapy, 2011. 13(6): p. 647-60.
163. Popp, F. C ., et al., Mesenchymal stem cells as immunomodulators after liver transplantation. Liver Transpl, 2009. 15(10): p. 1192-8.
164. Friedenstein, A. J., R. K. Chailakhjan, and K. S. Lalykina, The development of fibroblast colonies in monolayer cultures of guinea-pig bone marrow and spleen cells. Cell Tissue Kinet, 1970. 3(4): p. 393-403.
165. Friedenstein, A. J., et al., Stromal cells responsible for transferring the microenvironment of the hemopoietic tissues. Cloning in vitro and retransplantation in vivo. Transplantation, 1974. 17(4): p. 331-40.
166. Caplan, A. I., Molecular and cellular differentiation of muscle, cartilage, and bone in the developing limb. Prog Clin Biol Res, 1986. 217B: p. 307-18.
167. Caplan, A. I., Mesenchymal stem cells. J Orthop Res, 1991. 9(5): p. 641-50.
168. Kopen, G. C., D. J. Prockop, and D. G. Phinney, Marrow stromal cells migrate throughout forebrain and cerebellum, and they differentiate into astrocytes after injection into neonatal mouse brains. Proc Natl Acad Sci U S A, 1999. 96(19): p. 10711-6.
169. Petersen, B. E., et al., Bone marrow as a potential source of hepatic oval cells. Science, 1999. 284(5417): p. 1168-70.
170. Teratani, T., et al., Direct hepatic fate specification from mouse embryonic stem cells. Hepatology, 2005. 41(4): p. 836-46.
171. Le Blanc, K. and O. Ringden, Immunomodulation by mesenchymal stem cells and clinical experience. J Intern Med, 2007. 262(5): p. 509-25.
172. Keyser, K. A., K. E. Beagles, and H. P. Kiem, Comparison of mesenchymal stem cells from different tissues to suppress T-cell activation. Cell Transplant, 2007. 16(5): p. 555-62.
173. Le Blanc, K., et al., Mesenchymal stem cells for treatment of steroid-resistant, severe, acute graft-versus-host disease: a phase II study. Lancet, 2008. 371(9624): p. 1579-86.
174. Ning, H., et al., The correlation between cotransplantation of mesenchymal stem cells and higher recurrence rate in hematologic malignancy patients: outcome of a pilot clinical study. Leukemia, 2008. 22(3): p. 593-9.
175. Ball, L., et al., Third party mesenchymal stromal cell infusions fail to induce tissue repair despite successful control of severe grade IV acute graft-versus-host disease in a child with juvenile myelo-monocytic leukemia. Leukemia, 2008. 22(6): p. 1256-7.
176. Ringden, O., et al., Mesenchymal stem cells for treatment of therapy-resistant graft-versus-host disease. Transplantation, 2006. 81(10): p. 1390-7.
177. Le Blanc, K., et al., Treatment of severe acute graft-versus-host disease with third party haploidentical mesenchymal stem cells. Lancet, 2004. 363(9419): p. 1439-41.
178. Muller, I., et al., Application of multipotent mesenchymal stromal cells in pediatric patients following allogeneic stem cell transplantation. Blood Cells Mol Dis, 2008. 40(1): p. 25-32.
179. Horwitz, E. M., et al., Isolated allogeneic bone marrow-derived mesenchymal cells engraft and stimulate growth in children with osteogenesis imperfecta: Implications for cell therapy of bone. Proc Natl Acad Sci U S A, 2002. 99(13): p. 8932-7.
180. Koc, O. N., et al., Allogeneic mesenchymal stem cell infusion for treatment of metachromatic leukodystrophy (MLD) and Hurler syndrome (MPS-IH). Bone Marrow Transplant, 2002. 30(4): p. 215-22.
181. Le Blanc, K., et al., Transplantation of mesenchymal stem cells to enhance engraftment of hematopoietic stem cells. Leukemia, 2007. 21(8): p. 1733-8.
182. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.
183. Ball, L. M., et al., Cotransplantation of ex vivo expanded mesenchymal stem cells accelerates lymphocyte recovery and may reduce the risk of graft failure in haploidentical hematopoietic stem-cell transplantation. Blood, 2007. 110(7): p. 2764-7.
184. www.osiristx.com/pdf/PR%2039%2025Mar07%20Provacel%20Positive%20Results.pdf.
185. Kurtzberg, J., et al., Allogeneic human mesenchymal stem cell therapy (remestemcel-L, Prochymal) as a rescue agent for severe refractory acute graft-versus-host disease in pediatric patients. Biol Blood Marrow Transplant, 2014. 20(2): p. 229-35.
186. Kellathur, S. N. and H. X. Lou, Cell and tissue therapy regulation: worldwide status and harmonization. Biologicals, 2012. 40(3): p. 222-4.
187. Ahmadi, M., et al., Bone marrow mesenchymal stem cells and their conditioned media could potentially ameliorate ovalbumin-induced asthmatic changes. Biomed Pharmacother, 2016. 85: p. 28-40.
188. Nemunaitis, J., et al., Human marrow stromal cells: response to interleukin-6 (IL-6) and control of IL-6 expression. Blood, 1989. 74(6): p. 1929-35.
189. Sadovnikova, E. Y., et al., Induction of hematopoietic microenvironment by the extracellular matrix from long-term bone marrow cultures. Ann Hematol, 1991. 62(5): p. 160-4.
190. Lazarus, H. M., et al., Ex vivo expansion and subsequent infusion of human bone marrow-derived stromal progenitor cells (mesenchymal progenitor cells): implications for therapeutic use. Bone Marrow Transplant, 1995. 16(4): p. 557-64.
191. Yoo, J. U., et al., The chondrogenic potential of human bone-marrow-derived mesenchymal progenitor cells. J Bone Joint Surg Am, 1998. 80(12): p. 1745-57.
192. Fleming, J. E., Jr., et al., Monoclonal antibody against adult marrow-derived mesenchymal stem cells recognizes developing vasculature in embryonic human skin. Dev Dyn, 1998. 212(1): p. 119-32.
193. Ghilzon, R., C. A. McCulloch, and R. Zohar, Stromal mesenchymal progenitor cells. Leuk Lymphoma, 1999. 32(3-4): p. 211-21.
194. De Cesaris, V., et al., Isolation, proliferation and characterization of endometrial canine stem cells. Reprod Domest Anim, 2016.
195. Van Pham, P., et al., Isolation and proliferation of umbilical cord tissue derived mesenchymal stem cells for clinical applications. Cell Tissue Bank, 2016. 17(2): p. 289-302.
196. Zhao, G., et al., Large-scale expansion of Wharton's jelly-derived mesenchymal stem cells on gelatin microbeads, with retention of self-renewal and multipotency characteristics and the capacity for enhancing skin wound healing. Stem Cell Res Ther, 2015. 6: p. 38.
197. Huang, P., et al., Differentiation of human umbilical cord Wharton's jelly-derived mesenchymal stem cells into germ-like cells in vitro. J Cell Biochem, 2010. 109(4): p. 747-54.
198. Wang, S. J., et al., Chondrogenic Potential of Peripheral Blood Derived Mesenchymal Stem Cells Seeded on Demineralized Cancellous Bone Scaffolds. Sci Rep, 2016. 6: p.
199. Fazeli, Z., M. D. Omrani, and S. M. Ghaderian, CD29/CD184 expression analysis provides a signature for identification of neuronal like cells differentiated from PBMSCs. Neurosci Lett, 2016. 630: p. 189-93.
200. Wu, G., et al., Osteogenesis of peripheral blood mesenchymal stem cells in self assembling peptide nanofiber for healing critical size calvarial bony defect. Sci Rep, 2015. 5: p. 16681.
201. Shaer, A., et al., Isolation and characterization of Human Mesenchymal Stromal Cells Derived from Placental Decidua Basalis; Umbilical cord Wharton's Jelly and Amniotic Membrane. Pak J Med Sci, 2014. 30(5): p. 1022-6.
202. Fu, W. L., C. Y. Zhou, and J. K. Yu, A new source of mesenchymal stem cells for articular cartilage repair: MSCs derived from mobilized peripheral blood share similar biological characteristics in vitro and chondrogenesis in vivo as MSCs from bone marrow in a rabbit model. Am J Sports Med, 2014. 42(3): p. 592-601.
203. El-Badawy, A., et al., Adipose Stem Cells Display Higher Regenerative Capacities and More Adaptable Electro-Kinetic Properties Compared to Bone Marrow-Derived Mesenchymal Stromal Cells. Sci Rep, 2016. 6: p. 37801.
204. Plock, J. A., et al., The Influence of Timing and Frequency of Adipose-Derived Mesenchymal Stem Cell Therapy on Immunomodulation Outcomes After Vascularized Composite Allotransplantation. Transplantation, 2017. 101(1): p. el-ell.

1. A method of producing a CD4 expressing T cell which possesses regenerative activity while concurrently being able to inhibit immunological responses.

2. The method of claim 1, wherein said T cell is derived from a pluripotent stem cell.

3. The method of claim 2, wherein said pluripotent stem cell is selected from a group of stem cells comprising of: a) inducible pluripotent stem cells; b) embryonic stem cells; c) parthenogenic derived stem cells; and d) somatic cell nuclear transfer derived stem cells.

4. The method of claim 1, wherein said T cell expresses the transcription factor FoxP3.

5. The method of claim 1, wherein said inhibition of immunological responses is suppression of T cell proliferation.

6. The method of claim 5, wherein said T cell proliferation is induced by an agent selected from one or more agents from a group comprising of: a) a mitogen; b) a cytokine; c) a molecule capable of activating T cell receptor in an antigen nonspecific manner; and d) a molecule capable of activating T cell receptor in an antigen specific manner.

7. The method of claim 1, wherein said regenerative properties are ability to stimulate angiogenesis.

8. The method of claim 7, wherein said angiogenesis is creation of collateral circulation.

9. The method of claim 8, wherein said collateral circulation is used to treat ischemic conditions.

10. The method of claim 9, wherein said ischemic conditions are associated with activation of hypoxia inducible factor-1.

11. The method of claim 8, wherein said angiogenesis is associated with production of VEGF.

12. The method of claim 8, wherein said angiogenesis is associated with mobilization of endothelial progenitor cells.

13. The method of claim 8, wherein said angiogenesis is associated with mobilization of mesenchymal stem cells.

14. The method of claim 8, wherein said angiogenesis is associated with mobilization of hematopoietic stem cells.

15. The method of claim 1, wherein said regeneration involves activation of endogenous stem cells or progenitor cells.

16. The method of claim 15, wherein said endogenous stem cells or progenitor cells are capable of differentiating into tissue that is injured or hypofunctional.

17. The method of claim 15, wherein said endogenous stem cells or progenitor cells are mitotically quiescent in their basal state.

18. The method of claim 15, wherein said endogenous stem cells or progenitor cells express aldehyde dehydrogenase.

19. The method of claim 15, wherein said endogenous stem cells or progenitor cells express stem cell antigen-1.

20. The method of claim 15, wherein said endogenous stem cells or progenitor cells express interleukin-1 receptor.
